(19) United States
(12) Patent Application Publication
(10) Pub. No.: US 2010/0057010 A1
(43) Pub. Date: Mar. 4, 2010

(54) Fixation Device for Holding a Medical Instrument

(75) Inventor: Magnus Göransson, Malmo (SE)

Correspondence Address: COHEN, PONTANI, LIEBERMAN & PAVANE LLP, 551 FIFTH AVENUE, SUITE 1210, NEW YORK, NY, 10176, US

(73) Assignee: Unomedical A/S, Birkerod (DK)

(21) Appl. No.: 12/376,897

(22) PCT Filed: Apr. 20, 2007
(86) PCT No.: PCT/EP07/03473

§ 371 Date: Aug. 7, 2009

(30) Foreign Application Priority Data
Aug. 10, 2006 (EP) 06118749.8

(51) Int. Cl. A61B 17/34

(52) U.S. Cl. 604/164.04

(57) ABSTRACT

A fixation device for holding a medical instrument such as a trocar, on the skin of a patient in relation to surgery. An attachment device is provided for releasable fixation of the device on a patient and having an aperture through which the instrument may be received. A retention device is provided for frictionally engaging the instrument in the fixation device in a predetermined position; wherein the attachment device includes a flexible sheet-like member and the aperture is provided with an associated instrument receiving slot in the attachment device for radial insertion of the instrument. Such fixation device is suitable for use in relation to instruments such as trocars having a range of external diameters. The fixation device is advantageous since it is non-invasive. Moreover, the fixation device ensures an airtight seal with the trocar and provides for adjustment of the position of the trocar after the trocar is positioned without having to remove the entire trocar.

The present invention relates to a fixation device for holding a medical instrument, such as a trocar, on the skin of a patient in relation to surgery, said device comprising attachment means for releasably fixation of the device on a patient and having an aperture through which the instrument may be received; and retention means for frictionally engagement of the medical instrument in the fixation device in a predetermined position.

From U.S. Pat. No. 5,267,970 a device for anchoring a trocar sleeve to a patient's body is known. The anchoring device can be attached to the sleeve when this has been inserted into the patient's body, and as the longitudinal position of the anchoring device relative to the sleeve can be adjusted, the positioning of the sleeve is easy.

A fixation device is known from U.S. Pat. No. 5,792,112. In relation to surgery it is often used to insert one or more trocars, which is a tubular device through which instruments, such as camera equipment and surgical instruments of different kinds may be inserted. Moreover, it is normal that one of theses trocars are used for inflate the stomach with carbondioxide to create an elevated pressure, whereby it is easier for the surgeon to perform the surgery needed as a cavity is hereby created.

One of the drawbacks of the trocars is that they may be displaced just as there is a risk of leakage in the area where the trocar or surgical instrument in general penetrate the skin, since the skin of the patient usually is provided with a small slitting before the trocar is penetrated through the skin.

From U.S. Pat. No. 5,792,112,a trocar fixation devise is known which is capable of holding a trocar when it is placed through the skin of the patient. However, this device is not properly sealing the opening in relation to the trocar skin penetration. Moreover, a separate fixation device must be used for trocars having different external diameters.

From U.S. Pat. No. 5,397,335 a trocar assembly with adapter seal is known. However, this assembly does not address the issue of sealing around the outside of the trocar fixation device.

All patent and non-patent references cited in the present application are also hereby incorporated by reference in their entirety.

Another drawback of the trocars is that when the protective sphere surrounding the human body is penetrated, there is a risk of infections entering the body. It is therefore important that hygiene issues are taken into consideration when trocar assisted investigations are performed especially in relation to the openings made for inserting the trocar sleeve but also in relation to openings used to fixate the sleeve.

On this background it is an object of the invention to provide a fixation device for a trocar or the like which ensures a better degree of sealing and which can be used for trocars or other surgical medical instruments of different diameters.

These objects are achieved by a fixation device of the initially mentioned kind, wherein the set of operating members are pivotably arranged and pivotable between a first position for opening device and a second position for closing the operating members around the annular member of the device.

By such fixation device, the object is achieved by a device which is suitable for use in relation to instruments such as trocars having a range of external diameters. The device according to the invention is advantageous since it is non-invasive. Moreover, the device according to the invention ensures an airtight seal between the fixation device and the trocar as the clamping forces are evenly distributed around the annular contact area between the trocar and the instrument. The fixation device according to the invention is also advantageous by allowing for an adjustment of the position of the trocar after the trocar is positioned without having to remove the entire trocar. By the operating members being hinged they are swung backwards after having clamped the trocar onto the instrument and locked in a position, so that the operating members encompass the instrument and thereby do not protrude radially into the space surrounding the instrument above the trocar, whereby the risk of accidentally dislocating the trocar by unintentionally catching the operating arms of the trocar with tubes, wires, etc. used by the surgeon or other medical staff when using the trocar during their work.

According the invention, there is provided a fixation device for holding a trocar or similar surgical instrument in a fixed position, where the fixation device can be positioned into place after the trocar has been inserted. This makes it easy to mount the trocar, as it becomes possible to position the trocar accurately on the patient and then mount the fixation device to ensure the trocar is kept in its position during the surgery.

In the preferred embodiment of the invention, the fixation device is provided with the retention means which comprises an openable ring having two operating arms having cooperating locking means at their distal ends, and wherein said arms are pivotably joined to the exterior of the ring in such a manner that when the arms are joined together, the ring is closed and the locking means of the arms are engaged. This embodiment is advantageous as a precise locking mechanism is provided in a simple and efficient manner. A precise placement may be provided and subsequently the locking of the fixation device around the instrument can then be performed. This means that the user does not have to counteract any spring force when positioning the fixation device. The sheet member is flexible and allowed to wrinkle when the fixation device is “open” for positioning around the instrument.

In a first preferred embodiment, the retention means comprises a openable annular member which is operable by a set of operating members, so that the openable annular member is moved from between a first position where the ring is closed around the medical instrument and a second position where the inner ring member is open for attachment or detachment of the device around the instrument. Accordingly, the inner ring member opening is preferably aligned with the instrument receiving slot in the flexible sheet member. Hereby, the fixation device is functioning as a clamp with a sealing inner ring, such as a constriction member, which can be mounted around the medical instrument by inserting the instrument through the receiving slot and into the aperture.

The operating members are a pair of tabs attached to the openable ring in such a manner that the tabs are moveable relative to each other for operating said ring. Hereby, the operating member is provided with a clamping action for gripping around the cylindrical instrument, such as the trocar, which can be positioned and adjusted by two fingers. The fixation device according to the invention allows for an adjustment of the position of the trocar after the trocar is positioned without having to remove the entire trocar.

In an embodiment of the invention, the flexible sheet-like member may be provided with a first adhesive in an inner section around the aperture and a second adhesive in an outer section surrounding the inner section. Preferably, the first adhesive layer in said inner section comprises at least one hydrocolloid substance which when brought into contact with fluids forms a watertight gel thereby efficiently sealing the region where said medical instrument enters said patients body from the surrounding environment. Hereby, a high level of hygiene is ensured by the presence of the hydrocolloid substance around the region where the trocar sleeve has penetrated the skin of the patient's body.

In the preferred embodiment, the attachment means include a flexible sheet member and an adhesive flexible sheet material substantially surrounding the sheet member. Moreover, the inner ring member of the retention means may be permanently mounted on the flexible sheet member. Hereby, a simple and reliable design is achieved which may be secured to the patient's skin as a plaster ensuring advantage of providing a non-invasive trocar fixation device.

Preferably, the constriction member is made of a resilient relatively soft material, such as soft silicone. Hereby, a grip which also seals the device around the instrument is achieved.

Preferably, the operating member is made of a bendable hard plastic material, and which is adapted to performing a clamping action for holding the instrument. Hereby, the clamping action may be provided by a single part whereby the amount of parts and thereby the production costs may be reduced and reliability of the device may be increased.

In a second embodiment of a device according to the invention, the retention means include a first retention member which is integrally formed in the flexible sheet member as an annular protrusion having a first cone concentric with the aperture and a second retention member, which is an upper member having a protrusion having a second cone with a form corresponding to that of the first retention member, and which is positionable onto the sheet member, whereby the cones constrict around the instrument in the aperture.

Hereby, a simple and inexpensive fixation device is provided, where the device is mounted around the instrument via the instrument receiving slot of the attachment means and into the aperture in the first retention means. The device is subsequently firmly secured around the instrument by providing the second retention means on top of the first retention means, whereby the innermost of the two cones of the first and second retention members constricts around the instrument and retains the instrument in the device by friction.

In a preferred embodiment of this second embodiment, the membrane is disposed adjacent the flexible sheet member and hinged or otherwise foldably attached thereto. The flexible sheet member is provided with adhesive underneath for attaching to the patient's skin and the upper member is folded up for providing the retention. Accordingly, the upper member is preferably provided with a slot extending from the centrally disposed aperture and away from the fold line in an orientation perpendicular to said fold line. Accordingly, the receiving slot in the adhesive sheet member and the slot in the upper member may be provided with mutually substantially perpendicular orientations. However, it is realised by the invention that the functionality of this second embodiment is ensured as long as the two slots are differently oriented.

The invention is described in more detail with reference to the accompanying drawings, in which:

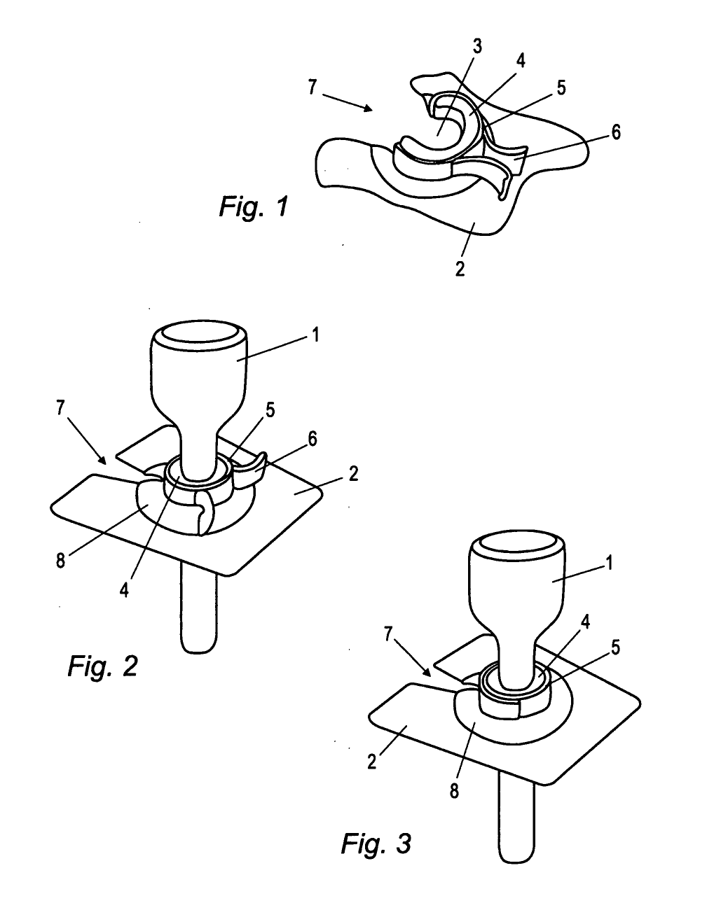

FIG. 1 is a perspective view of a fixation device according to a first embodiment of the invention;

FIG. 2 is a perspective view of the fixation device of FIG. 1 clamped around a trocar;

FIG. 3 is a perspective view of the fixation device of FIG. 1 clamped and locked around a trocar;

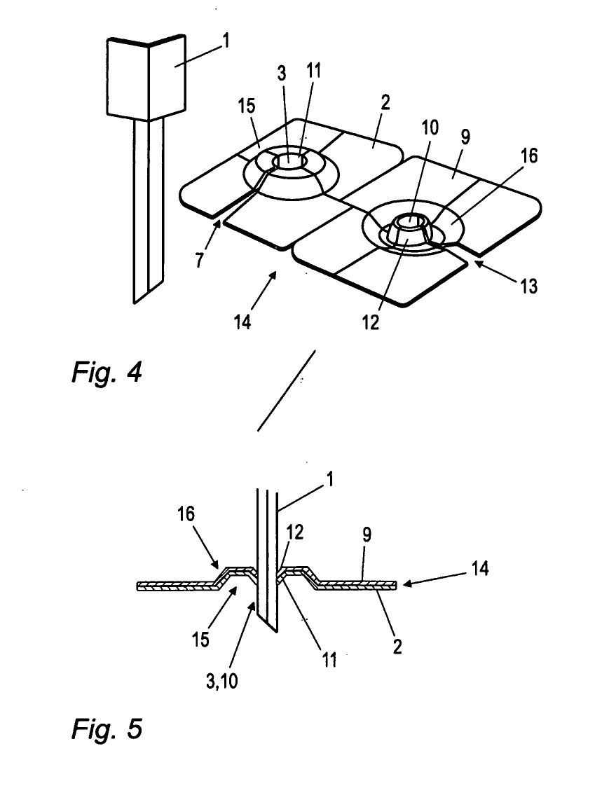

FIG. 4 is a perspective view of a fixation device according to a second embodiment;

FIG. 5 is a cross-sectional side view of the fixation device in FIG. 4;

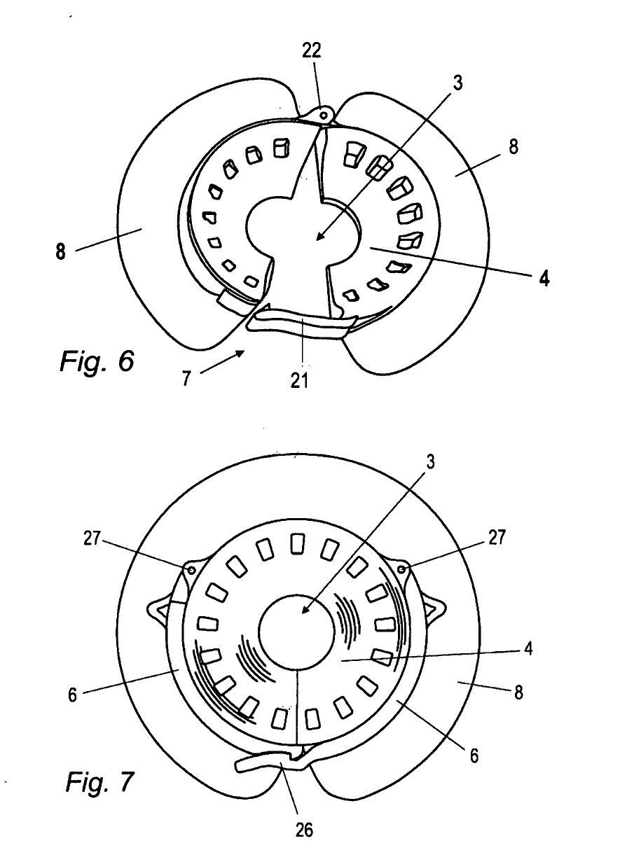

FIG. 6 is a perspective top view of a third embodiment of a fixation device according to the invention,

FIG. 7 is a top view of a fourth embodiment of a fixation device according to the invention, and

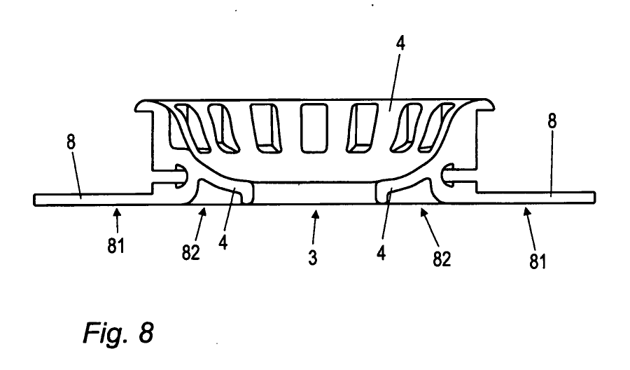

FIG. 8 is a cross-section view of the fixation device of FIG. 6 or 7.

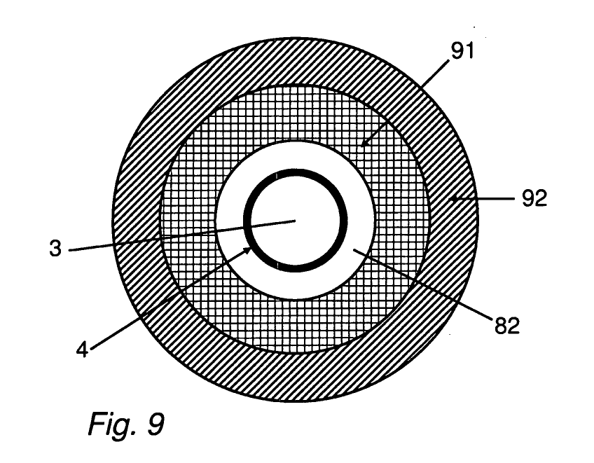

FIG. 9 is a bottom view of a preferred embodiment of the invention

With reference to FIGS. 1 to 3, a first embodiment of the fixation device according to the invention is shown. As shown in FIG. 1, the device comprises a ring-like constriction member 5 with a pair of operation wings 6 for opening the constriction member. This outer ring 5 is made of a hard, bendable plastic material suitable for providing a clamping action for clamping the device around the medical instrument 1. Inside the outer ring there is provided an openable ring 4 which is made of a soft material, such as a foam or similar resilient material for establishing a gas-tight seal around the medical instrument 1 when the ring is clamped around this instrument 1. The resilient inner ring 4 is capable of adapting its shape in accordance with the diameter of the instrument thereby making the device suitable for different external diameters of the trocar or other instrument 1.

The inner and outer ring members 4, 5 are opened by pressing the two tabs 6 towards each other whereby the instrument insertion slot 7 is opened. The instrument insertion slot 7 is provided on the flexible sheet 2 opposite the operating tabs 6 and providing passage from the outer periphery of the sheet member 2 into the centrally disposed aperture 3. When the trocar 1 is inserted into the aperture 3, the pressure on the tabs 6 is released and the elasticity of the outer ring 5 causes the ring members 4, 5 to spring back and close around the inserted trocar or similar medical instrument 1, as shown in FIG. 2.

The retention means may comprise means for providing a substantially uniform pressure directed radially from the circumference inward towards said medical instrument (1).

As a further feature, the tabs 6 may be suitably shaped with a curved shape and hinged so that the tabs 6 can be pushed towards the receiving slot after having clamped the instrument 1 and hereby lock the device in the position clamped and sealed around the instrument 1. The device may be produced as a disposable device and accordingly the locking mechanism need not be relockable but could be a permanent locking of the device around the trocar 1.

The ring member 5 is mounted on an annular flexible support 8 which is provided on a flexible sheet member 2 which has an adhesive on the underside for adhesively securing the fixation device to the patient's skin and around the trocar. It is appreciated that it is irrespective whether the device according to the invention is mounted on the patient before or after the trocar is placed as the fixation device is non-invasive. The adhesive could alternatively be provided on the upper side or provided on a hydrocolloid sheet material.

With reference to FIGS. 4 and 5 a second embodiment of a fixation device according to the invention is shown. According to this embodiment, the device is made with a foldable upper member 9 adjacent the flexible sheet member 2, which in this embodiment preferably is substantially rectangular in shape and more preferably substantially square. The flexible sheet member 2 is provided with an adhesive on its underside.

The flexible sheet member 2 is provided with an instrument receiving slot 7 providing a passage from the outside of the sheet member 2 and into the aperture 3 which is centrally placed in the sheet member 2. Around the aperture 3 an annular protrusion is provided concentrically with the aperture 3. On the inside of this annular protrusion 15, there is provided a cone portion 11 which tapers downwards and defines the aperture 3.

Along a different side edge of the sheet member 2 than where the insertion slot 7 is provided, there is provided a fold line 14 dividing the sheet member 2 and the upper member 9. The upper member 9 serves as a retention member cooperating with the protrusion 15 and in particular the cone portion 11 of the sheet member 2 for constricting around and thereby gripping the trocar 1.

The upper member 9 is provided with a central aperture 10 and a slot 13 with a direction substantially perpendicular to the fold line creating a passage from the aperture 10 to the side edge opposite the fold line. Around the aperture 10, a second protrusion 16 mirroring the first protrusion 15 of the sheet member 2 is provided. This second protrusion 16 includes a cone portion 12 corresponding to the first cone portion 11 of the sheet member 2. The upper member 9 is so dimensioned that the second protrusion including the second cone portion 12 fits onto the first protrusion 15 and the first cone portion 11 when the upper member 9 is folded onto the sheet member 2 along the fold line 14.

After the sheet member 2 is placed around the trocar 1 by sliding the trocar 1 through the receiving slot 7 and into the aperture 3, the upper member 9 is folded onto the sheet member 2. The upper member slot 13 ensures that the upper member 9 passes the trocar 1 so that the upper member 9 is placed with its protrusion 16, cone portion 12 and aperture 10 concentrically arranged on top of the corresponding items 3, 11, 15 of the sheet member 2. As the second cone 12 is fitted inside and pressed against the first cone 11, the second cone portion 12 is constricted and thereby performs a clamping action on the trocar 1. By providing at least the upper member 9 in a relative resilient material a firm and sealed fixation of the trocar in the device may be achieved. Preferably, the upper member 9 could be provided with an adhesive for locking the upper member 9 in its folded position.

As shown in FIGS. 6 and 7, embodiments of the invention are provided wherein the sheet-like attachment member 8 is divided into two adjacent portions that are mutually pivotally joined by the retention means.

In FIG. 6 is shown a third embodiment of the fixation device, wherein the retention means comprises an openable ring 4 having two portions corresponding to the two adjacent portions of the flexible attachment means 8. The ring 4 is provided with a hinge 22 on the exterior side of the ring and at a first end and a locking member 21 at the opposite end thereof.

In FIG. 7, a fourth embodiment is shown, wherein the retention means comprises an openable ring 4 having two operating arms 6 having cooperating locking means 26 at their distal ends, and wherein said arms 6 are pivotably joined to the exterior of the ring 4 in such a manner that when the arms 6 are joined together, the ring 4 is closed and the locking means 26 of the arms 6 are engaged. The flexible sheet 8 in this fourth embodiment may be one piece like the embodiment in FIG. 1. However, it is realised that the embodiment shown in FIG. 1 could alternatively be in two pieces. In the embodiment of FIG. 7, the sheet member 8 may be flexible and allowed to wrinkle when the fixation device is “open” for positioning around the instrument.

The arms 6 are at their first ends pivotably mounted via a hinge 27 to the annular ring member 4, 5 symmetrically around the insertion slot 7. The arms 6 are hereby pivotable between a first position, where they function as opening levers when pressed together (see FIGS. 1 and 2), and a second position where the arms 6 encompass the ring member 4, 5. The arms 6 are shaped with a curvature so that the arms 6 follow the circular curvature of the ring member 4, 5.

For both the embodiments shown in FIGS. 6 and 7, a precise placement may be provided and subsequently the locking of the fixation device around the instrument can then be performed. This means that the user can easily counteract any spring force when positioning the fixation device. The sheet member 8 may be provided with hydrocolloid or the like on its underside 81 for adhesive attachment to the skin of the patient.

In FIG. 8 there is shown a cross-section of an embodiment of a fixation device according to the embodiments shown in FIGS. 6 and 7. According to the embodiment of FIG. 8, the adhesive underside 81 is not extending all the way into the aperture 3 which is defined by the ring 4. Instead the ring member 4 is formed with an annular recess 82 around the innermost portion of the ring 4. This provides an improved flexibility when the innermost cone portion of the ring 4 is impacted by a multiple of trocars so that the ring 4 is able to be deformed depending on the amount (or size) of trocars that are placed in the fixation device.

In FIG. 9 there is shown a bottom view of a preferred embodiment of a fixation device according to the invention. An inner section 91 with one adhesive is surrounded by an outer section 92 comprising a second adhesive. The adhesive in the inner section 91 is preferable an adhesive hydrocolloid. The adhesive in the outer section is preferable a skin friendly substance which holds the fixation device in place, while the hydrocolloid ensures a watertight seal. The aperture 3 of the fixation device is defined by the ring member 4 in a way similar to that described in FIG. 8. Also shown is an annular recess 82.

Above, the invention is described with reference to some preferred embodiments. However, it is realised that other embodiments may be provided without departing from the scope of the invention as defined in the accompanying claims.

1-28. (canceled)

29. A fixation device for holding a medical instrument such as a trocar, on the skin of a patient in relation to surgery, said device comprising:
attachment means for adhesive fixation of the device to the skin of the patient, said attachment means comprising a flexible sheet-like member with receiving slot and a flexible support with an aperture through which the instrument may be received; and
retention means for providing frictional engagement between the flexible support and the medical instrument in a predetermined position, said retention means comprises an openable annular member which is operable by a set of operating members for clamping the device around the instrument;
wherein the set of operating members are pivotably arranged and pivotable between a first position for opening the device and a second position for closing the operating members around the openable annular member of the device.

30. A fixation device according claim 29, wherein the operating members comprises means for locking the device in the second position clamped and sealed around the instrument said means for locking being activated by pushing the operating members towards the receiving slot after having clamped the device around the instrument.

31. A fixation device according claim 29, wherein the operating members are hinged to the openable annular member at a first end opposite a distal end.

32. A fixation device according claim 31, wherein locking means are provided at the distal end of the operating members.

33. A fixation device according to claim 29, wherein the openable annular member comprises an outer ring and an inner ring.

34. A fixation device according claim 33, wherein the outer ring is made of a hard material suitable for providing a spring force for a clamping action around the instrument and the inner ring is made of is made of a resilient relatively soft material, such as soft silicone or thermopolyethylene (TPE).

35. A fixation device according to claim 29, wherein the openable annular member has pivotably attached two operating arms having cooperating locking means at their distal ends, and wherein said arms are pivotably joined to the exterior of the annular member in such a manner that when the arms are joined together, the annular member is closed and the locking means of the arms are engaged.

36. A fixation device according to claim 29, wherein the openable annular member has two portions corresponding to the two adjacent portions of the flexible attachment means and wherein said annular member is provided with a hinge at a first end and a locking member at the opposite end.

37. A fixation device according to claim 29, wherein the flexible sheet-like-member is adhesive and substantially surrounds the flexible support.

38. A fixation device according to claim 33, wherein the inner ring of the retention means is permanently mounted on the flexible support.

39. A fixation device according to claim 37, wherein the flexible sheet-like member is provided with a first adhesive in an inner section around the aperture and a second adhesive in an outer section surrounding the inner section.

40. A fixation device according to claim 37, wherein the inner ring and the flexible support are integrally formed.

41. A fixation device according to claim 37, wherein the flexible support is divided into two adjacent portions that are mutually pivotally joined by the retention means.

42. A fixation device according to claim 29, wherein said retention means comprises means for providing a substantially uniform pressure directed radially from the circumference inward towards said medical instrument.

43. A fixation device according to claim 29, wherein the operating members are made of a bendable hard plastic material, and which is adapted to performing a clamping action for holding the instrument.

44. A fixation device according to claim 29, wherein the flexible sheet-like member is provided with a first adhesive in an inner section around the aperture and a second adhesive in an outer section surrounding the inner section.

45. A fixation device according to claim 44, wherein said first adhesive in said inner section comprises at least one hydrocolloid substance.

46. A fixation device according to claim 29, wherein the aperture associated with the receiving slot in the attachment means for radial insertion of the instrument.

47. A fixation device for holding a medical instrument such as a trocar, on the skin of a patient in relation to surgery, said device comprising:
attachment means for adhesive fixation of the device to the skin of the patient, said attachment means comprising a flexible sheet-like member with an aperture through which the instrument may be received; and
retention means for frictionally engagement of the medical instrument in the fixation device in a predetermined position, wherein the retention means include a first retention member which is integrally formed in the flexible sheet-like member as an annular protrusion having a first cone concentric with the aperture and a second retention member, which is an upper member having a second protrusion having a second cone with a form corresponding to that of the first retention member, and which is positionable onto the flexible sheet-like member whereby the cones constrict around the instrument in the aperture.

48. A fixation device according to claim 47, wherein said upper member is disposed adjacent the flexible sheet-like member and hinged or otherwise foldably attached thereto along a fold line.

49. A fixation device according to claim 47, wherein the flexible sheet-like member is provided with an associated instrument receiving slot for radial insertion of the instrument.

50. A fixation device according to claim 48, wherein said upper member is provided with a slot extending from the centrally disposed aperture and away from the fold line in an orientation perpendicular to said fold line.

51. A fixation device according to claim 50, wherein the receiving slot and the slot in the upper member are provided with mutually substantially perpendicular orientations.

52. A fixation device according to claim 47, wherein the flexible sheet-like member is provided with a first adhesive in an inner section around the aperture and a second adhesive in an outer section surrounding the inner section,

53. A fixation device according to claim 52, wherein said first adhesive in said inner section comprises at least one hydrocolloid substance.

54. A fixation device for holding a medical instrument such as a trocar, on the skin of a patient in relation to surgery, said device comprising:
attachment means for adhesive fixation of the device to the skin of the patient, said attachment means comprising a flexible sheet-like member with an aperture through which the instrument may be received; and
retention means for frictionally engagement of the medical instrument in the fixation device in a predetermined position;
wherein the flexible sheet-like member is provided with a first adhesive in an inner section around the aperture and a second adhesive in an outer section surrounding the inner section.

55. A fixation device according to claim 54, wherein said first adhesive in said inner section comprises at least one hydrocolloid substance.

56. A fixation device according to claim 54, wherein the aperture is provided with an associated instrument receiving slot in the attachment means for radial insertion of the instrument.
